(19) United States
(12) Patent Application Publication
(10) Pub. No.: US 2008/0299632 A1
(43) Pub. Date: Dec. 4, 2008

(54) Methods for Recovering Oil from a Fractionated Dry Milling Process

(75) Inventors: David James Winsness, Alpharetta, GA (US); Greg Barlage, Sandston, VA (US)

Correspondence Address: CANTOR COLBURN, LLP, 20 Church Street, 22nd Floor, Hartford, CT, 06103, US

(73) Assignee: GS CLEANTECH CORPORATION, New York, NY (US)

(21) Appl. No.: 12/130,399

(22) Filed: May 30, 2008

Related U.S. Application Data
(60) Provisional application No. 60/941,121, filed on May 31, 2007.

(51) Int. Cl. C12P 7/10; C07C 29/74

(52) U.S. Cl. 435/165; 568/913

(57) ABSTRACT

Processes for recovering oil from thin stillage produced during a fractionation-based dry milling process. The thin stillage can be heated or heated in combination with centrifuging to separate and recover oil from the thin stillage. Optionally, the thin stillage may be concentrated prior to recovering the oil.

### CROSS REFERENCE TO RELATED APPLICATIONS

This application claims the benefit of U.S. Provisional Patent Application No. 60/941,121, filed on May 31, 2007, incorporated herein by reference in its entirety.

### BACKGROUND

The present disclosure generally relates to producing oil from grains, and more particularly, to recovering oil from a co-product of a fractionated dry milling process used to form ethanol from the grains.

Over the past thirty years, significant attention has been given to the production of ethyl alcohol, or “ethanol,” for use as an alternative fuel. Ethanol not only burns cleaner than fossil fuels, but also can be produced using grains such as corn, a renewable source. Ethanol can be produced from various grains such as corn by either a wet milling or a dry mill process. In the wet milling process, the corn kernel is separated into different components such as germ, starch, protein, and fiber, resulting in several co-products. For example, separated germ is further processed for oil recovery; starch is saccharified and fermented for ethanol production; and protein and fiber can be used as feed material. In a dry mill process, the corn is not fractionated and only one co-product is produced in addition to ethanol. In this process, the entire ground corn kernel is processed through fermentation and distillation, where the end products are ethanol and whole stillage. The whole stillage contains water, a portion of starch that was not fermented, and the remaining non-fermentable portions of the kernel of corn such as protein, fiber, corn oil and ash. Water is then removed from the whole stillage to form the dried distillers grains. At present, an estimated one hundred “dry milling” plants are producing over 4 billion gallons of ethanol per year. Additional plants presently under construction are expected to add more than two billion gallons to this total. Technology exists today that effectively recovers corn oil from the whole stillage from these dry mill ethanol facilities.

While most of the ethanol production facilities currently in use are considered “dry milling”, there has been a recent movement to build “fractionation-based” dry milling ethanol production facilities. These fractionated facilities attempt to separate as much of the non-fermentable portions of the grain as practical prior to the fermentation step. For example, corn kernels are comprised of three primary components: endosperm, germ, and bran. The endosperm contains the majority of the starch within the kernel of corn, or about 95%, whereas the germ and the bran contain high concentrations of non-fermentables (fiber, protein, and corn oil). Wet and dry fractionation technologies exist today that can be integrated into the dry milling process to effectively separate the endosperm, germ, and bran with minimal losses. The separated endosperm can then be conveyed to the fermentation process, and the germ and bran can then be sold directly to other markets.

With less non-fermentable mass entering the ethanol dry milling production process, greater volumes of ethanol can to be produced per volume of fermentation capacity. In addition, separating non-fermentables prior to fermentation allows for a reduced mass of whole stillage exiting distillation and advantageously reduces energy loads on the whole stillage dehydration equipment. The downside of current technology is that the separation equipment and processes used need improvements to make the processes economically viable. For example, some of the starch exits with the non-fermentable components, thereby increasing the mass of corn required per volume of ethanol produced. This is satisfactory as long as the co-products retain favorable value and ethanol production capacity increases relative to the reduced non-fermentables in the process.

Today, in both traditional dry milling and fractionated dry milling ethanol production facilities, the whole stillage can be dehydrated by separating the heavy phase from the lighter phase using a centrifuge. The heavier phase is referred to as wet distillers grains and the lighter phase is referred to as thin stillage. The thin stillage is concentrated efficiently using multi-effect evaporation to produce a product referred to as condensed distillers solubles. Because the non-fermentables were previously separated from the starch during the fractionation based dry milling process, it was previously believed that any oil obtained from the thin stillage would be minimal so no efforts have been made to recover oil therefrom.

### BRIEF SUMMARY

Disclosed herein are processes for recovering oil from thin stillage produced in a fractionation-based dry milling process. In one embodiment, the process includes fractionating the whole grain; separating a portion of the lesser starch fractions from the higher starch fractions; fermenting the higher starch fractions to produce ethanol; distilling the fermented fraction to separate the ethanol from the fermented fraction and produce ethanol and whole stillage; further separating the whole stillage into thin stillage and a wet cake; and recovering oil from the thin stillage.

In another embodiment, a fractionation-based dry milling process comprises fractionating grain into a least one high starch fraction and at least one low starch fraction; fermenting and distilling the at least one high starch fraction to produce ethanol and whole stillage; separating the whole stillage to produce thin stillage and wet distillers grains; heating the thin stillage at a temperature greater than 212° F. and at a pressure greater than its vapor pressure to prevent boiling of the thin stillage during the heating; cooling the thin stillage to a temperature less than 212° F.; and separating oil from the thin stillage.

The disclosure may be understood more readily by reference to the following detailed description of the various features of the disclosure and the examples included therein.

### BRIEF DESCRIPTION OF THE DRAWINGS

Referring now to the figures wherein the like elements are numbered alike:

FIG. 1 schematically illustrates a prior art non-fractionation-based dry milling process based on processing 18,000,000 bushels of corn per year and expected compositional yields; and

FIG. 2 schematically illustrates a fractionation-based dry milling process based on processing 18,000,000 bushels of corn per year and expected compositional yields.

### DETAILED DESCRIPTION

Disclosed herein are processes for recovering oil from a fractionation-based dry mill ethanol production process, also sometimes referred to as a fractionated dry grind process. The process generally includes fractionating the grain to separate the non-fermentables from the starch fraction. The starch fraction is then subjected to fermentation and distillation to produce ethanol, leaving behind whole stillage. The whole stillage is then further processed to produce wet distillers grains and thin stillage. As is known in the art, mechanical separation techniques can be utilized to effect separation of the thin stillage from the wet distillers grains using, for example, a press/extruder, a decanter centrifuge (also simply known as a “decanter”), or a screen centrifuge. As will be discussed in greater detail below, the thin stillage is then processed to recover oil therefrom. Applicants have surprisingly discovered that current fractionation-based dry milling processes produce thin stillage having an extractable oil content that is about equal to the oil content contained within thin stillage produced during a non-fractionated process. The recovered oil can be sold as high value feed or fuel stock. Alternatively, the oil can be converted to biodiesel.

The fractionation-based dry mill process can be applied to various grains such as corn, rice, sorghum (i.e., milo), wheat, barley, oat, rye, and the like. For ease of understanding, corn is referred to below for the purpose of illustration but should not be considered limiting. It should also be noted that the particular fractionation process steps are not intended to be limited to any particular method. Suitable fractionation-based dry milling processes for separating the starch fraction from the non-fermentable fractions are generally described in International Application No. WO2006055489A2, US Patent Publication No. 20050118693A1, incorporated herein by reference in their entireties.

In one embodiment, the process generally includes fractionating the grain into various components generally defined by fractions containing substantial amounts of fermentable starch and those that do not have substantial amounts of fermentable starch but rather contain substantial amounts of non-fermentable components (e.g., bran, germ, and/or the like), i.e., separating lesser starch fractions from the higher starch fractions. As used herein, lesser starch fraction means less than 50% starch content, with less than 30% starch content in some embodiments, and less than 10% starch content in still other embodiments. Higher starch content refers to a starch content greater than 50%, with greater than 70 percent starch content is some embodiments, and with greater than 90% starch content in still other embodiments. By way of example, the fractionating process applied to corn generally includes separating corn into one or more low starch fractions consisting essentially of bran and/or germ, and a high starch fraction consisting essentially of the endosperm. The higher starch fraction is then fermented and distilled to produce ethanol, leaving behind whole stillage. The germ and/or bran fractions can be further processed in a conventional manner to produce feed, oil, or the like. In the present disclosure, the whole stillage obtained after fermentation and distillation of the higher starch fraction is further separated into thin stillage and a wet cake, also referred to as wet distillers grains (WDG). The thin stillage is then subjected to an oil recovery process to remove and recover oil from the thin stillage.

When comparing the wet distillers grains and thin stillage produced at traditional and fractionation-based corn dry mill production facilities, Applicants have unexpectedly discovered marginal differences in oil content as they both contain roughly 35% of the total oil that was contained in the corn. However, Applicants have discovered substantial differences in the WDG. The fractionation-based corn dry mill process eliminates a substantial volume of fiber, protein, and corn oil from the WDG. Specifically, it has been discovered that about 50% of the total corn oil is removed during fractionation and contained within the germ and bran fractions leaving only about 15% of the total corn oil within the wet distillers grains.

In one embodiment, the oil recovery process from a fractionation-based dry mill ethanol production facility includes separating the WDG from the whole stillage so as to produce thin stillage. Next, the thin stillage is subjected to a heating step or a heating step in combination with centrifuging. The heating step is used to free the oil from within the emulsified thin stillage to allow the oil to be released from the emulsion and be recovered using a gravity separation or by way of the centrifuge (gravity separation using a non-hermetically sealed centrifuge or settling tank may be used after the heating step but performance is enhanced through the use of a centrifuge). In one embodiment, the heating step includes heating to a temperature greater than 212° F. while simultaneously pressurizing the thin stillage to prevent boiling. In another embodiment, the heating step includes heating to about 230° F. to about 250° F. and at a pressure above its vapor pressure so as to prevent boiling. The thin stillage is then allowed to cool to below 212° F. prior to recovering the oil. Heating can be effected in any heat exchanger such as a wide gap plate and frame heat exchanger, shell and tube heat exchanger, scraped surface plate and frame heat exchanger, and the like or by way of indirect or direct steam injection. By way of example, the thin stillage can be configured to flow into a feed heater system through an interchanger, wherein the thin stillage inlet feed is preheated. The temperature is then raised to greater than 212° F. and a backpressure valve is maintained as an elevated pressure such that the pressure is above the vapor pressure of the thin stillage, e.g., 40 psi, such that boiling of the heated thin stillage is prevented. Prior to recovering the oil via separation using the gravity separator, the thin stillage is preferably cooled. By use of the interchanger, the return heated thin stillage can be cooled. After cooling, the thus processed thin stillage can be gravity separated or may be centrifuged to recover the oil. Centrifuging can be effected by introducing the thin stillage into a horizontal decanter centrifuger, a vertical disk centrifuger, or the like.

In another embodiment, a three phase centrifuge is used in combination with heat to process the whole stillage. In this manner, the whole stillage can be separated into 3 products: a light phase (oil and emulsion), a medium phase (water and dissolved solids) and a heavy phase (solids or wet cake). The light phase can then be further processed using an oil dryer, evaporator, heating step, cooling step, and/or centrifuge so as to recover the oil contained therein.

In another embodiment, the oil recovery process includes concentrating the thin stillage using existing evaporators within the fractionated-based dry mill ethanol production facility. The concentrate can have moisture content greater than about 10% to less than about 85%. The concentrated thin stillage is then subjected to heating as described above. The oil can be recovered from the concentrate by passing it through a centrifuge (e.g., a self cleaning bowl type) or by gravity separation as previously described. In one embodiment, the concentrate is fed to a disk stack centrifuge at a temperature between about 150° F. and 300° F. and a pH between about 3 and 6. Suitable disk centrifuges include those commercially available from Alfa Laval under the trade names 510, 513, and 617. In an exemplary embodiment, the concentrated thin stillage is concentrated to about 20% solids and heated to a temperature of about 240° F. for about 30 minutes and then cooled through the use of an interchanger or the like to about 205° F. prior to being delivered to the centrifuge or a gravity separation device. By allowing the thin stillage to be concentrated first in the ethanol facilities existing evaporator prior to attempting to recover any oil, a reduction in total volume of material is realized (substantial quantities of water can be removed by the evaporator) and some of the emulsion has been broken through the heat treatment and evaporation process.

In another embodiment, a filtration apparatus such as pressurized membrane separation unit is used to separate oil and various other components within the thin stillage (i.e., components such as solubles, sterols, and the like). Suitable filtration apparatuses are disclosed in U.S. Pat. No. 5,250,182, the disclosure of which is incorporated by reference in its entirety.

Once the oil has been reduced within the thin stillage, it may be desirable to dehydrate this product independently. Traditionally, the material is sold on a wet basis or blended with the wet distillers grains (WDG) because it is generally difficult to dehydrate with high levels of oil concentrations. Removal of oil as described herein allows for more efficient dehydration methods such as spray drying or ring drying, allowing for the first time the production of defatted distillers solubles. Scrapped surface evaporators may benefit from the energy efficiency of the drying process to remove as much water as possible using multi-effect evaporative drying prior to final drying. Suitable evaporators include, without limitation, single or multi-effect evaporators.

As previously discussed, the percentage of total oil contained within the WDG is considerably less in a “fractionated” dry grind ethanol facility as most of this volume is removed during germ separation. While the volume is considerably less, it may still be desirable to further reduce the oil concentration of this WDG. Reduced oil concentrations are beneficial to some poultry, fish, and livestock feed rations and the extracted oil is also beneficial to biodiesel and other production industries. In one embodiment, the wet cake is rehydrated. For example, the wet cake exiting the two phase or three phase centrifuges can be rehydrated using water, thin stillage, or the light phase exiting the three phase centrifuge. This rehydrated wet cake is then centrifuged in a two or three phase centrifuge. Optionally, heat may be applied during the centrifuge process. The process may be repeated as may be desired for some applications. The rehydration and multi-centrifugation process releases a portion of the oil that is bound within the wet cake and this oil can then be recovered by way of any of the techniques described within the methods for oil recovery from thin stillage or condensed solubles.

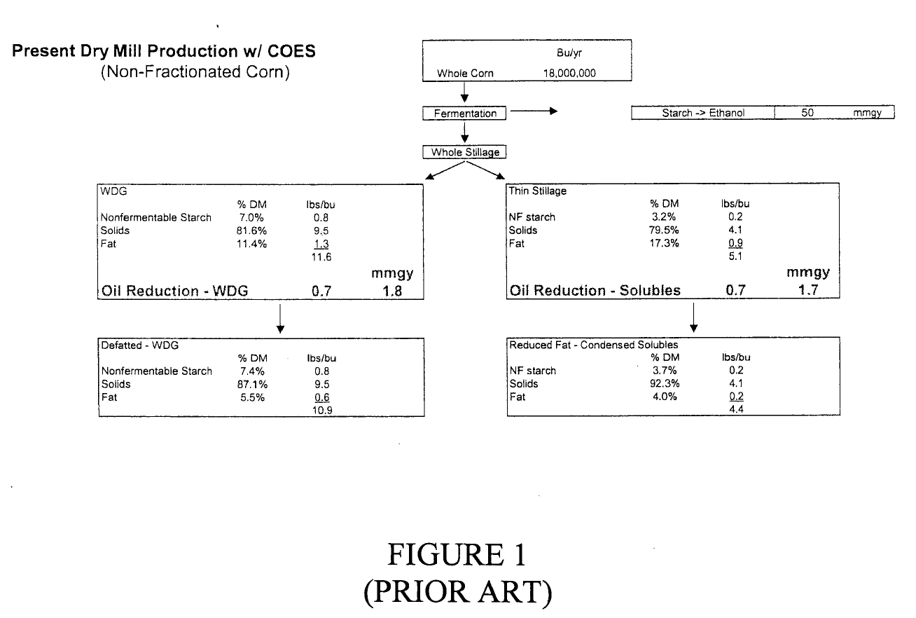

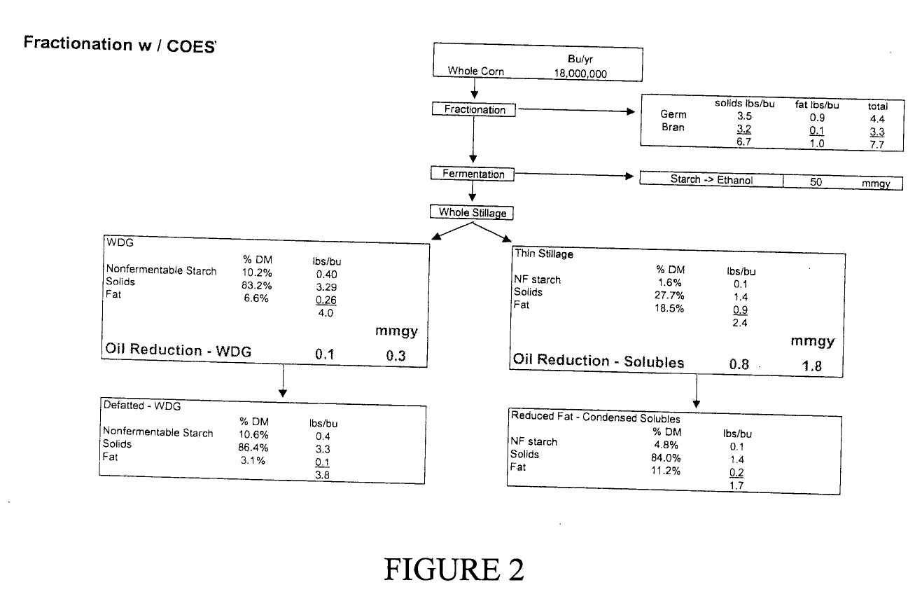

FIGS. 1 and 2 provide a comparison of corn oil recovery yields for a non-fractionated-based dry mill process compared to a fractionated-based dry mill process. As shown in FIG. 1, current non-fractionated-based dry mill processes generally include processing entire ground corn kernel through fermentation and distillation, where the end products are ethanol and whole stillage. The whole stillage is then separated into thin stillage and wet distillers grains. Separation can include mechanical separation such as with a centrifugal decanter. The thin stillage and the wet distillers grains can then be further processed to recover oil contained therein. For example, based on 18 million bushels of corn per year, the dry mill ethanol production facility is expected to yield 50 million gallons per year (mmgy) of ethanol during the fermentation of the starch. The recovered oil from the thin stillage yield is about 1.7 mmgy and from the wet distillers grains the recovered oil is about 1.8 mmgy. (Although in this instance, the amount recovered was 1.8 mmgy, it has generally been found that the amounts recovered from WDG can vary from about 0.8 to about 1.8 mmgy, but is generally greater than 1.1 mmgy.

FIG. 2 illustrates the expected yields for a fractionation-based dry mill process based on processing 18 mmgy bushels of corn per year. As previously discussed, fractionation separates the non-fermentables such as germ and bran prior to fermentation of the starch. Fractionation does not affect ethanol yield during fermentation, which remains at about 50 mmgy. However, the recovery of oil from the thin stillage was unexpectedly equivalent to the non-fractionated dry milling process at about 1.8 mmgy compared to 1.7 mmgy as noted above for the non-fractionated process. Where the fractionation-based dry milling process significantly differs is the amount of oil recovered from the WDG, which was about 0.3 mmgy compared to 1.8 mmgy for the non-fractionated dry milling process. It was previously believed that fractionation-based dry milling process resulted in negligible amounts of non-fermentables contained in the whole stillage that would render any oil recovery commercially impractical. However, that clearly and unexpectedly was not the case. The processes as described in Applicants disclosure above provide a means for recovering significant amounts of additional oil in a cost effective manner.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed:

1. A method of processing whole grains used for producing ethanol comprising:
fractionating the whole grain to form low starch fractions and higher starch fractions;
fermenting the higher starch fractions to produce ethanol and whole stillage;
distilling the fermented fraction to separate the ethanol from the whole stillage;
further separating the whole stillage into thin stillage and a wet cake; and
recovering oil from the thin stillage.

2. The method of claim 1, wherein the grain is corn.

3. The method of claim 1, wherein recovering the oil from the thin stillage comprises filtering the thin stillage in a pressurized membrane separation unit to separate and recover the oil.

4. The method of claim 1, wherein recovering the oil from the thin stillage comprises centrifuging the thin stillage.

5. The method of claim 1, wherein recovering the oil from the thin stillage comprises heating the thin stillage at a temperature and pressure effective to separate and recover the oil.

6. The method of claim 5, further comprising centrifuging the thin stillage after heating.

7. The method of claim 6, wherein centrifuging the thin stillage to separate and recover the oil comprises introducing the thin stillage into a horizontal decanter centrifuge.

8. The method of claim 6, wherein centrifuging the thin stillage to separate and recover the oil comprises introducing the thin stillage into a vertical disk stack centrifuge.

9. The method of claim 1, wherein recovering the oil from the thin stillage comprises concentrating the thin stillage to form a concentrated thin stillage; and heating the concentrated thin stillage at a temperature and pressure effective to separate and recover the oil.

10. The method of claim 9, further comprising centrifuging the thin stillage after heating.

11. The method of claim 9, wherein concentrating the thin stillage comprises filtering the thin stillage in a pressurized membrane separation unit to separate and recover the oil.

12. The method of claim 9, wherein concentrating the thin stillage comprises evaporating water from the thin stillage.

13. The method of claim 1, wherein recovering the oil from the thin stillage comprises concentrating the thin stillage; heating the thin stillage; and centrifuging the thin stillage to separate and recover the oil.

14. The method of claim 13, wherein concentrating the thin stillage comprises filtering the thin stillage in a pressurized membrane separation unit to separate and recover the oil.

15. The method of claim 13, wherein concentrating the thin stillage comprises evaporating water from the thin stillage.

16. The method of claim 13, wherein centrifuging the thin stillage to separate and recover the oil comprises introducing the thin stillage into a vertical disk stack centrifuge.

17. The method of claim 13, wherein centrifuging the thin stillage to separate and recover the oil comprises introducing the thin stillage into a horizontal decanter centrifuge.

18. The method of claim 13, wherein heating the thin stillage comprises introducing the thin stillage to a heat exchanger.

19. The method of claim 1, wherein heating the thin stillage comprises heating the thin stillage to a temperature greater than 212° F. at a pressure above the vapor pressure of the thin stillage so as to prevent boiling; and cooling the thin stillage to a temperature less than 212° F.

20. The method of claim 1, further comprising dehydrating the thin stillage subsequent to recovering the oil from the thin stillage to produce defatted distillers solubles.

21. The method of claim 9, further comprising dehydrating the thin stillage subsequent to recovering the oil from the thin stillage to produce defatted distillers solubles.

22. A fractionation-based dry milling process, the process comprising:
fractionating grain into a least one high starch fraction and at least one low starch fraction;
fermenting and distilling the at least one high starch fraction to produce ethanol and whole stillage;
separating the whole stillage to produce thin stillage and wet distillers grains; and
heating the thin stillage at a temperature greater than 212° F. and at a vapor pressure effective to prevent boiling of the thin stillage during the heating;
cooling the thin stillage to a temperature less than 212° F.; and
separating oil from the thin stillage.

23. The fractionation-based dry milling process of claim 22, wherein the grain is corn.

24. The fractionation-based dry milling process of claim 22, further comprising centrifuging the thin stillage subsequent to cooling the thin stillage.

25. The fractionation-based dry milling process of claim 24, further comprising concentrating the thin stillage prior to heating the thin stillage.

26. The fractionation-based dry milling process of claim 22, further comprising dehydrating the thin stillage subsequent to recovering the oil from the thin stillage to produce defatted distillers solubles.
